(19) United States
(12) Patent Application Publication
(10) Pub. No.: US 2011/0076737 A1
(43) Pub. Date: Mar. 31, 2011

(54) CARRIERS FOR IMMOBILIZATION AND REUSABILITY OF ENZYMES

(76) Inventor: Magdy Mahmoud Mustafa Elnashar, Cairo (EG)

(21) Appl. No.: 12/866,748

(22) PCT Filed: Jun. 11, 2009
(86) PCT No.: PCT/EG09/00016

§ 371 Date: Aug. 9, 2010

(30) Foreign Application Priority Data
Jun. 22, 2008 (EG) 2008061064

(51) Int. Cl. C12N 11/10; C07K 17/12

(52) U.S. Cl. 435/178; 530/350; 530/387.5

(57) ABSTRACT

This patent involves improvement of the thermal stability of natural polymers, “hydrogels”, to be used as supports for covalent immobilization of enzymes. The presence of the new functionality has been proved by FTIR. In brief, the new polymeric carrier has the main following advantages: • Natural polymer & friendly to the environment • Highly hydrophilic • Containing active groups for covalent immobilization of enzymes& antibodies Thermally stable (35-95° C.) Porous The modified carrier could be used for immobilization of enzymes on the industrial scale. The immobilized enzymes could be reused for tens of times, which will reduce the cost of the enzymes and their products.

### TECHNICAL FIELD

Polymers (Biopolymers) and Biochemistry (Enzymes).

### BACKGROUND ART

Alginates and carrageenans polymers have been used as carriers for immobilization of enzymes and cells using physical techniques such as the adsorption, entrapment and encapsulation methods. As these polymers are enjoying many advantages, for examples they are hydrophilic, environmentally friendly and porous. To increase the rigidity of alginates and carrageenans (poly anions) after containing the enzymes and cells, their surfaces were treated with cationic polymers and with a di- or poly-aldehyde.

However, on the industrial level, the treated alginates and carrageenans (hydrogels) by these methods are not used on the industrial level due to the following:

1- The drawback of the non covalent bond such as adsorption or entrapment as they are weak bonds and consequently the active entrapped or adsorbed material (e.g. enzyme) is usually leaked from the polymers after the first use.

2- The mechanical and thermal stability of the non treated hydrogels melt or dissolve at low temperature and moderate reaction conditions.

### DISCLOSURE OF INVENTION

The alginates and carrageenans are very abundant polymers in nature; FDA approved and could be shaped in many forms such as films, disks and beads. These properties were encouraging to use them on the industrial scale after some treatment. Based on the fact that these polymers are polyanionic, they could be treated with a polycation such as chitosan and polyimines. It is known that the deacetylation of chitin to chitosan resulted into chitosan with 5-15% amide and 85-95% amine groups. Thus the 85-95% free amino groups were used to form a strong polyelectrolyte with the gel. The strength of this polyelectrolyte interaction is as strong as the covalent bone). Thus, the novelty of this work is treatment of alginates and carrageenans polymers with a cationic polymer such as chitosan or polyimines then immobilizing the enzymes to the hydrogels via covalent bonds using their free amino groups through a mediator such as di-aldehyde. As a result of this treatment:

1- The thermal stability of the gels was improved from 35° C. to 95° C.

2- New functionalities have been incorporated to the treated gel with chitosan or polyimines and the di-aldehyde such as the aldehyde group, which was proved by the Infrared spectroscopy (IR). The new aldehydic's carbonyl group, which came from the reaction of the glutaraldehyde with the chitosan or the polyimines on the gel has been proved by the Infra Red (IR) technique at a wavelength of 1720 cm⁻¹ in case of carrageenan gel and at 1647 cm⁻¹ for the alginate, which differs than that of the chitosan's amide group at 1668 cm⁻¹ or that of the pure alginate at 1600 cm⁻¹.

3- Few enzymes have been successfully immobilized covalently to the gels.

4- As a result of the strong covalent bond between the enzymes and the gels, the enzymes do not leak and the immobilized enzymes could be reused on the industrial scale for tens of times, which reduce both the cost of the enzyme and the enzymatic products.

The methodology of modifying the alginates and carrageenans gels for immobilization of enzymes was as follow:

1- The alginates and carrageenans gels' powders of 1-5% (w/v) were dissolved in distilled water for 1-5 hours at 25-80° C. till complete dissolution. The gels were prepared in form of sheets, disks or beads using the syringe or the Innotech Encapsulator.

2- The gels were soaked in 0.1-10% (w/v) chitosan or polyimines for 10 min to 8 hours to improve the gels' thermal stability.

3- The poly- or di-aldehyde compound to be used as a mediator between the gel and the enzyme to bind them via a covalent bond has been chosen to be glutaraldehyde, which was used at 1-10% (v/v) and for 1-5 hours.

4- The treated hydrogel with the active aldehyde groups were soaked in different enzymes for 1-16 hours to immobilize them with covalent bonds.

1- Hydrogels were treated to immobilize different proteins using covalent bonds to enable reusability of the proteins for tens of time, which is very important on the industrial scale to reduce the cost of the proteins.

2- The hydrogels in claims 1 could be natural polysaccharides, which are alginates and carrageenans at a concentration of 0.1-5% (w/v) and especially 0.5-3% (w/v)

3- The proteins in claims 1 could be antibodies, antigens and enzymes. Different enzymes were used in this work.

4- The hydrogels in claim 1 could be in the form of sheets, disks and beads, especially beads and disks were used.

5- The gel disks and beads in claim 4 were treated with polycations to form polyelectrolyte interaction with the hydrogels.

6- The polycations in claim 5 were polyimines and chitosan.

7- The concentration of the polycations in claim 6 were used at a concentration of 0.1-10% (w/v) and for 10 min to 8 hours.

8- The duration of soaking in claim 7 was especially 1-5 hours and at 0.5-7% (w/v).

9- The treated hydrogels in claim 8 were soaked in 1-10% (v/v) poly or di-functionalized compounds.

10- The compounds in claim 9 were especially di-functional compound and especially glutaraldehyde.

11- The glutaraldehyde in claim 10 was used at a concentration of 2-5% (v/v) and for 1-5 hours.

12- The duration of incubation for the treated hydrogel in claim 11 was for 1.5-3.5 hours.

13- The activated hydrogels in claim 12 were proved using the Infrared spectroscopy (IR) to prove the incorporation of new functionalities, aldehyde groups.

14- The activated hydrogels with the free aldehyde groups in claim 12 were soaked in different enzymes for 1-16 hours for immobilization.
